(19) United States
(12) Patent Application Publication
(10) Pub. No.: US 2026/0210947 A1
(43) Pub. Date: Jul. 23, 2026

(54) SYSTEMS AND METHODS FOR DETECTION OF GLYPHOSATE

(71) Applicant: THE TRUSTEES OF INDIANA UNIVERSITY, Bloomington, IN (US)

(72) Inventors: Shahir S. RIZK, South Bend, IN (US); Nathanael Bryce SMITH, South Bend, IN (US); Adrian S. RIZK, South Bend, IN (US)

(21) Appl. No.: 19/456,628

(22) Filed: Jan. 22, 2026

Related U.S. Application Data
(60) Provisional application No. 63/748,772, filed on Jan. 23, 2025.

(51) Int. Cl. G01N 33/53; G01N 33/543

(52) U.S. Cl. CPC G01N 33/5308; G01N 33/54386; G01N 2333/245; G01N 2430/20

(57) ABSTRACT

The present disclosure relates to devices and methods for detecting glyphosate. More specifically, the present disclosure relates to a device comprising a fluorescently labeled glyphosate binding protein as a biosensor that is configured to change fluorescence as a function of glyphosate concentration, a first excitation source operable to excite the biosensor at a first wavelength, a light detector configured to collect emitted light, and a processor configured to process any difference in fluorescence before and after a sample is added to the biosensor.

### CROSS REFERENCE TO RELATED APPLICATIONS

This application claims priority to U.S. Provisional Patent Application No. 63/748,772 filed on Jan. 23, 2025, the disclosure of which is expressly incorporated herein.

### TECHNICAL FIELD

The present disclosure relates to systems and methods for measuring and detecting glyphosate.

### BACKGROUND

Glyphosate is the most commonly used herbicide in the United State with annual use reaching up to 300 million pounds. The herbicide can contaminate groundwater and pose potential health risks to humans. Therefore, it is important to develop effective, cheap, portable, and easy-to-use methods to detect glyphosate in soil and water samples.

There have been examples of low-cost fluorometer-like devices in the literature. For example, a device may use an Arduino processor, an LED as a light source, and a low-cost photoresistor as the light detector in benchtop set up. However, such devises include components that are not housed in an enclosed chamber, and therefore, lights must be turned off during measurements to minimize interference from ambient light. Additionally, such devices are not portable since the components are anchored to a work bench. Some portable fluorescence measurement devices use high powered light sources and therefore, require a heat sink to prevent overheating. Other similar devices are used as substitutes for fluorescent microscopes or for the detection of algae in water. However, there are no known devices that use biosensors for the detection of glyphosate.

The present disclosure is directed to the systems and methods of detecting glyphosate in water and soil samples. Specifically, the disclosure is directed to systems and methods of detecting glyphosate by implementing a biosensor protein and detecting changes in glyphosate based on fluorescence changes.

### SUMMARY

The present disclosure includes one or more of the features recited in the appended claims and/or the following features which, alone or in any combination, may comprise patentable subject matter.

According to a first aspect of the present disclosure, a device for detecting glyphosate, comprises a fluorescently labeled glyphosate binding protein as a biosensor, an excitation source with an appropriate wavelength to excite the biosensor, a light detector configured to collect emitted light, and a processor configured to process any difference in fluorescence before and after a sample is added to the biosensor.

In some embodiments, the device may be portable and self-contained. In some embodiments, the device may provide a binary result indicating the presence or absence of glyphosate. In some embodiments, the device may be configured to operate as a miniature fluorometer. In some embodiments, the biosensor may comprise E. coli bacterial periplasmic binding protein, PhnD. In some embodiments, the protein may be labeled with AlexaFluor488 or a fluorophore that changes fluorescence when glyphosate binds to the protein.

According to a second aspect of the present disclosure, a method for detecting glyphosate, comprises adding a sample to a portable device comprising a fluorescently labeled glyphosate binding protein, exciting the protein with an appropriate wavelength, collecting emitted light from the protein through a light detector, processing the collected light to determine any difference in fluorescence before and after a sample is added to the protein, and providing a binary result indicating the presence or absence of glyphosate.

In some embodiments, protein may include a biosensor comprising E. coli bacterial periplasmic binding protein, PhnD. In some embodiments, the protein may be labeled with AlexaFluor488. In some embodiments, the protein may be configured to change fluorescence as a function of glyphosate concentration in the sample.

### BRIEF DESCRIPTION OF DRAWINGS

The following description accompanies the drawing(s), all given by way of non-limiting examples that may be useful to understand how the described method and composition may be embodied.

FIG. 1 illustrates the conformational change associated with ligand binding equilibrium. When PhnD binds to its ligand, a hinge-bending conformational change take place and the protein goes from an open (left) to a closed (left) form. A cysteine at position 17 (shown as black spheres) is used to attached fluorescent groups. Binding of the ligand triggers changes in fluorescence.

FIG. 2A is a graph of a biosensor, PhnD177NΔ17C-AF488, showing a large increase in fluorescence (300%) when glyphosate is added. The plot shows the fluorescence spectra in the apo (unbound) form (open circles) and in the presence of 250 μM glyphosate (closed circles).

FIG. 2B is a graph showing titration of the biosensor with glyphosate. Monitoring the change on fluorescence as a function of glyphosate showed a dissociation constant (K_{D}) of 34 μM.

FIG. 3 is an illustration of a first embodiment of a device configured to detect glyphosate.

FIG. 4 is a graph showing the peak emission of the blue LED used as the excitation source for the device.

FIG. 5 is a graph show the result from test conducted on the device. Light output (fluorescence) of the biosensor was collected for 10 seconds, then 50 μL of the sample were added and light output was collected for an additional 10 seconds. Samples containing glyphosate (final concentrations of 170 μM) show a large increase in fluorescence (solid lines), whereas samples with no glyphosate (buffer) showed no increase in fluorescence (dotted lines).

FIG. 6 is a graph showing the ability of the device to detect the presence of commercial herbicides containing glyphosate. Baseline light output was collected for 10 seconds, then samples were added to the biosensors, followed by another light collection for 10 seconds. Each sample was done in triplicates. The data shows that tap water (solid grey line) shows no increase in signal, whereas the brand name herbicide (RoundUp concentrate; dotted black line) or the generic herbicide (Compare N Save; solid black line) show a large increase in light signal, indicating the presence of glyphosate. Both the generic and the brand name herbicides were diluted in tap water per manufacturer's instructions.

FIG. 7 is a graph showing the results of tests conducted with soil samples. Organic topsoil was treated with either tap water, nano pure (polished water), the generic herbicide, or the brand name herbicide. The samples were incubated overnight, then about 1 g of the soil was removed from each treatment, mixed with 1 mL working buffer, and filtered to remove debris. The device was used to test the filtered buffer extracts from each sample. While tap water (solid grey line) and nano pure water (solid black line) show no signal change, samples treated with the generic herbicide (black dotted line) or the brand name herbicide (grey dotted line) show a large change in signal indicating the presence of glyphosate.

FIG. 8 is a graph showing the result of tests conducted in the absence of protein and in the presence of maltose. A test was performed in the absence of the protein where glyphosate was added (solid grey line), resulting in no change in light output. To show that the protein is specific of glyphosate detection, a test was performed in the presence of the protein biosensor where maltose (and unrelated ligand) was added (black dotted line), resulting in no change in light output. A test for the detection of glyphosate was used as the positive control (black dotted line) showing an increase in light output when glyphosate was added.

FIG. 9 is an illustration of a second embodiment of a device configured to detect glyphosate.

FIG. 10 is an illustration of a bottom section of an enclosure for the device described in the present disclosure.

FIG. 11 is an illustration of a top section of an enclosure for the device described in the present disclosure.

FIG. 12 is an illustration of a cuvette holder located in the device described in the present disclosure.

FIG. 13 is an illustration of a lens holder located in the device described in the present disclosure.

FIG. 14 is an illustration of a lens holder wings located in the device described in the present disclosure.

FIG. 15 is an illustration of a lens housing located in the device described in the present disclosure.

FIG. 16 is a graph showing the change in fluorescence of the PBP164C/138A-coumarin biosensor upon addition of phosphate.

FIG. 17A is a graph showing the titration curves of PBP164C/138A in the absence of glyphosate.

FIG. 17B is a graph showing the titration curves of PBP164C/138A in the presence of 1 mM glyphosate.

FIG. 18 is a graph showing the change in fluorescence in the PBP (blue channel) and the PhnD (green channel) in response to the presence of RoundUp only (RU with no PO4), both RoundUp and phosphate present (RU with PO4), or only phosphate (PO4 only) when using a device with two sensor elements.

FIG. 19A is a graph showing lyophilized protein performance in response to fresh protein.

FIG. 19B is a graph showing lyophilized protein performance in response to glyphosate after 4 months of storage.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary.

As used herein, the term “patient” or “user” refers to any subject including mammals and humans. The patient may have a disease or suspected of having a disease and as such is being treated with a drug. In some instances, the patient is a mammal, such as a human, a premature neonate, neonate, infant, juvenile, adolescent, or adult thereof. In some instances, the term “patient,” as used herein, refers to a human (e.g., a man, a woman, or a child). In some instances, the term “patient,” as used herein, refers to laboratory animal of an animal model study. The patient or subject may be of any age, sex, or combination thereof.

The term “treating” refers to administering a therapy in an amount, manner, or mode effective (e.g., a therapeutic effect) to improve a condition, symptom, disorder, or parameter associated with a disorder, or a likelihood thereof.

The terms “essentially” or “substantially” as used herein mean to a great or significant extent, but not completely.

The term “about” as used herein refers to any values, including both integers and fractional components that are within a variation of up to +10% of the value modified by the term “about.”

The present disclosure is directed to the development and implementation of a portable system that can detect the presence of glyphosate in different samples. The present disclosure is directed to systems and methods that can successfully distinguished between glyphosate-contaminated and uncontaminated samples in water and soil samples, including those treated with commercial herbicides.

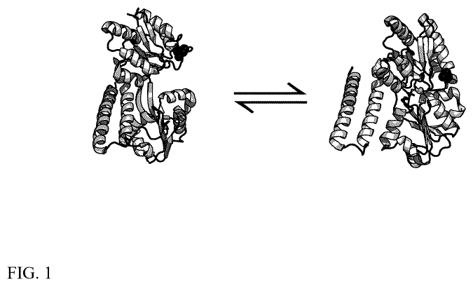

The present disclosure is directed to systems and methods for measuring glyphosate. An engineered Escherichia coli bacterial periplasmic binding protein, PhnD, can be reengineered to bind to glyphosate with high affinity and specificity. Mutation of glutamic acid 177 to asparagine as well as truncation of the last 6 amino acid residue may result in about a 150-fold increase in the affinity for glyphosate. Attachment of a thiol-reactive fluorescent group may be accomplished by the introduction of a unique cysteine at position 125. As shown in FIG. 1, upon ligand (e.g., glyphosate) binding, the protein undergoes a large conformational change, which in-turn triggers a change in fluorescence. In particular, when thiol-reactive acrylodan or coumarin are attached at position 125, a decrease in fluorescence is observed upon binding of the protein to glyphosate.

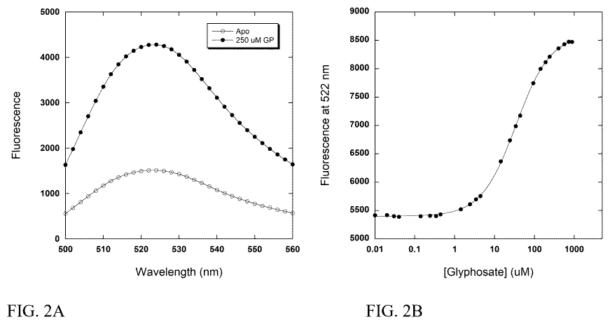

In one embodiment, different positions of a protein (e.g., PhnD) may be used for fluorophore attachment. Fluorophore attachments to the protein may produce an increase in fluorescence when the ligand (e.g., glyphosate) is bound to the protein. In one embodiment, the cysteine mutation is moved to position 17 (illustrated as a black sphere in FIG. 1) to produce a protein, PhnD177NΔ17C. PhnD177NΔ17C also comprises a C-terminal HSV tag followed by a 6-histidine tag. Purified PhnD177NΔ17C may be conjugated to one of five different fluorophores. The changes in fluorescence upon addition of glyphosate is illustrated in Table 1. A protein, PhnD177NΔ17C-AF488, formed when PhnD177NΔ17C is conjugated with AlexaFluor488 (AF488) shows the largest change in fluorescence with about a 180% increase in fluorescence when saturating concentrations of glyphosate is added (see FIG. 2A).

TABLE 1
Fluorescence changes of PhnD177NΔ17C
conjugates in response to glyphosate

| Fluorophore | % change | λmax (nm) |
|---|---|---|
| Acrylodan | −212 | 460 |
| Coumarin | −18 | 470 |
| AlexaFluor488 | +180 | 524 |
| TMR | 0 | N/A |
| Texas Red | −3 | 612 |

In one embodiment, the protein (e.g., PhnD, PhnD177NΔ17C, PhnD177NΔ17C-AF488) is configured to be a biosensor element that changes fluorescence as a function of glyphosate concentration. In one embodiment, PhnD177NΔ17C is conjugated to a fluorophore to be configured as a biosensor element that changes fluorescence as a function of glyphosate concentration. In one embodiment, PhnD177NΔ17C conjugated to AlexaFluor488 (PhnD177NΔ17C-AF488), is configured to be a biosensor element that changes fluorescence as a function of glyphosate concentration. In one embodiment, monitoring the change in fluorescence of PhnD177NΔ17C-AF488 as a function of glyphosate concentration may be used to determine a dissociation constant (KD) of PhnD177NΔ17C-AF488. The dissociation constant of PhnD177NΔ17C-AF488 was determined to be about 34 μM (FIG. 2B).

In one embodiment, a low-cost portable device 10 comprises a fluorescently labeled glyphosate binding protein as a biosensor element 18. In one embodiment, the biosensor element 18 may be PhnD177NΔ17C-AF488. The device 10 is configured to detect any change in fluorescence as an indication of the presence of glyphosate in a sample. In one embodiment, the device 10 is a self-contained and is an easy to use device for an untrained non-scientist end-user. In one embodiment, the device 10 is configured to provide a binary end result indicating presence or absence of glyphosate at the push of a button. In one embodiment, the device 10 is configured to retain sufficient information for an expert user to examine the raw data. In one embodiment, the device 10 is configured to operate as a miniature fluorometer 12 comprising an excitation source 14 with an appropriate wavelength to excite a conjugated fluorophore 16 on the biosensor element 18, a light detector 20 configured to collect the emitted light with sufficient sensitivity, and a processor 22 with the capability to process the difference in fluorescence before and after a sample is tested with the biosensor element 18.

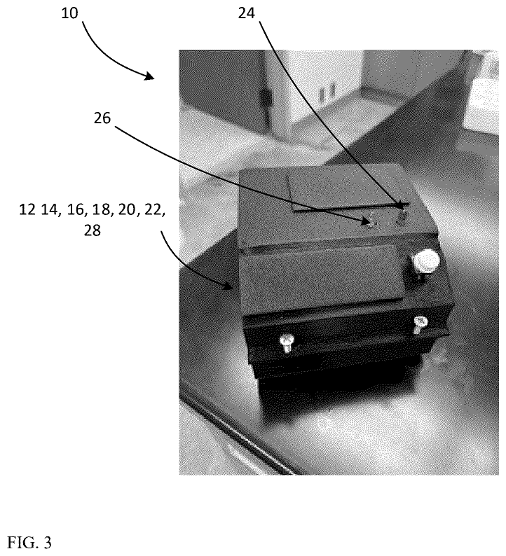

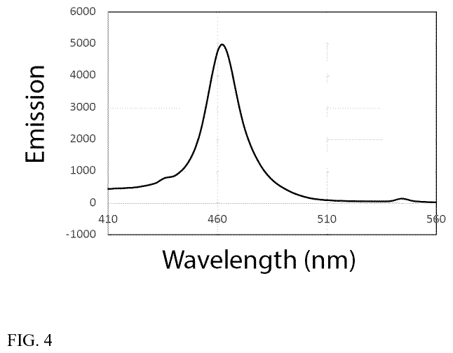

In one embodiment, the device 10 is a self-contained 3-D printed chamber that houses all of the components described above. The device 10 may be configured to block ambient light from interfering with the measurements. One embodiment of the device 10 is illustrated in FIG. 3. In one embodiment, the device 10 comprises a blue LED 14 with the peak emission at 462 nm (see FIG. 4) as the excitation source 14. In other embodiments, the device 10 may comprise different LEDs as the excitation source 14. In one embodiment, the device 10 may comprise a TSL2591 photo diode sensor as the light detector 20. The device 10 may comprise a sensitive light detector 20 with a wide dynamic range between 188 μLux up to 88,000 Lux. The light detector 20 may be a AS7341 10-channel light sensor 20, a AS7262 6-channel visible light sensor 20, or a TCS34725 RGB sensor 20.

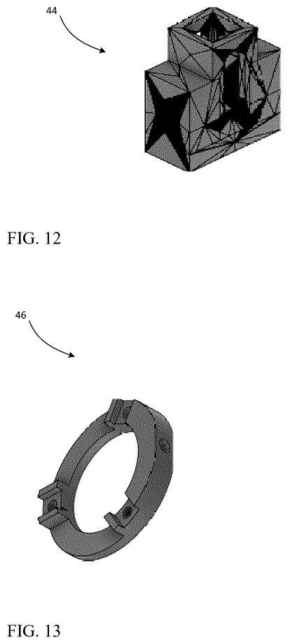

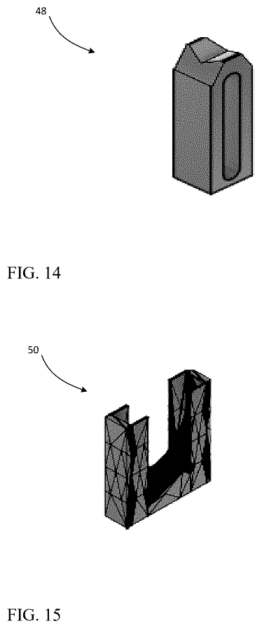

In one embodiment, the device 10 may be a 3-D printed chamber that houses a four-sided clear cuvette in a cuvette holder 44 (see FIG. 12), a light detector 20, and a LED 14 positioned at a 90° angle relative to the light detector 20. The device 10 may also include a 3D printed lens housing 50 for a small lens positioned between the sample and the light detector 20 in a lens holder 46 with lens wings 48 (see FIGS. 13-15) to focus the emitted light. In one embodiment, the device 10 may process the information by an Arduino Nano (or a similar Arduino-type processor) using a custom-made program that orchestrates data collection, manipulation, and data output. In one embodiment, the device 10 may include a slot 28 for attaching an optional LCD screen (LCD 1602 module). If the screen is not used, a 3D printed cover may be used to block ambient light.

In one embodiment, a device 10 comprising a single excitation LED source 14 and the TSL2591 light sensor as the light detector 20 may be sufficient for detecting changes in light intensity because the TSL2591 light sensor is operable to detect total light (Lux value) emitted from a sample. By measuring the initial lux value, and the lux value after addition of the sample, any increase in the lux value may be attributed to glyphosate binding, which is associated with an increase in fluorescence.

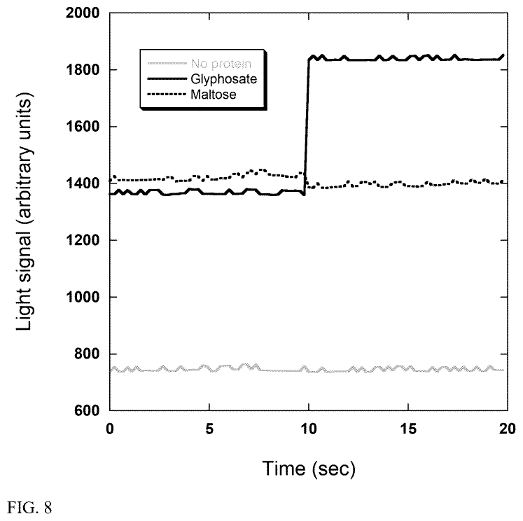

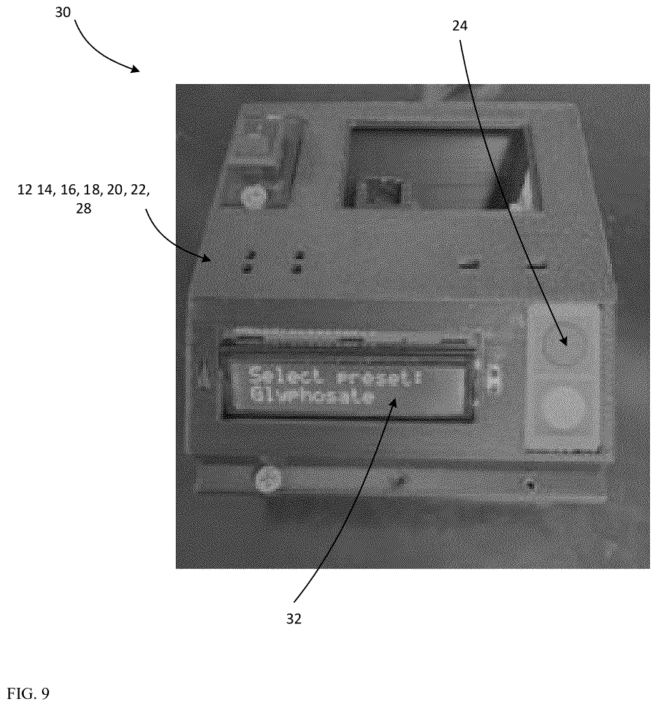

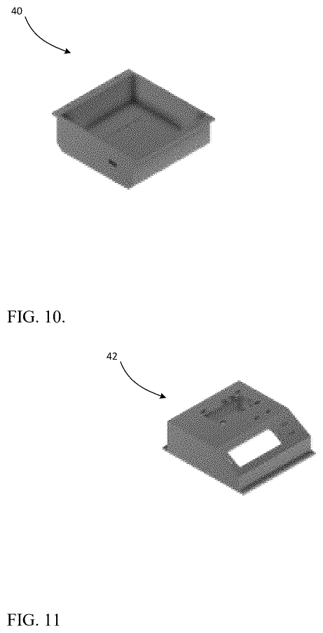

However, the TSL2591 light sensor is operable to only detect total light intensity changes without distinguishing between different wavelengths. Therefore, in some embodiments, a device 30, as shown in FIG. 9, may include components that enable simultaneous detection of fluorescence at multiple wavelengths. The device 30 includes additional slots 28 for up to 3 different LED light sources 14 for excitation of the sample near a sample holding chamber. The device 30 includes a AS7341 multichannel light sensor as the light detector 20. The AS7341 multichannel light sensor can detect up to 10 independent light channels (415 nm, 445 nm, 480 nm, 515 nm, 555 nm, 590 nm, 630 nm, 680 nm, IR, and total light). The device 30 includes a screen 32 (e.g., a LCD screen) for better interaction with the user. The screen 32 may provide detailed instructions on sample handling and testing, show raw data as it is collected by the device 30, and/or provide final results of the test (e.g., a label indicating contaminated or safe). The device 30 may be powered by a power source such as 9-volt battery as the power source. FIGS. 10 and 11 illustrate a bottom section of the device 30 and a top section of the device 30.

In one embodiment, a cuvette, containing 200 to 300 nM PhnD177NΔ17C-AF488 (e.g., biosensor element 18) in 3 mL of working buffer (50 mM MOPS, 150 mM NaCl, pH 6.9) may be placed into the cuvette holder 44 (see FIG. 12) within the device 10, 30. Then a cover may be placed over the top of the device 10, 30 to block ambient light. A first button may be selected to trigger the LED 14 (e.g., blue LED) to excite the biosensor element 18, and emission data received by the light detector may be collected frequently in certain time intervals. In some embodiments, the emission data may be collected every 200 milliseconds over the period of 10 seconds. This establishes a “blank” baseline for fluorescence. A first average and a first standard deviation may be determined by the processor 22. Then the sample suspected of containing glyphosate may be added to the cuvette (typically 50 μL) and mixed. A second button may be selected to trigger LED 14 excitation of the biosensor element 18 and emission data received by the light detector 20 may be collected for another time period (e.g., for a 10-second time period). A second average and a second standard deviation may then be calculated.

The processor 22 may be configured to compare the first and the second average by dividing the baseline after addition of sample by the average of a “blank” sample. If the ratio of the two averages lies between a predetermined range (e.g., 1.1-3), then the sample may be designated as contaminated with glyphosate and a binary output indicating contamination may be provided. For example, a red light 24 on the outside of the device 10 may be turned on to indicate contamination. If the ratio of the two averages is below the predetermined range, then the sample may be designated as clear of contamination and a binary output indicating non-contamination may be provided. For example, a green light 26 on the outside of the device 10 may be turned on to indicate non-contamination. If the ratio is greater than the pre-determined range, a binary output indicating a faulty test may be provided. For example, both the green and the red lights may be turned on to indicate a faulty test.

In one embodiment, the present disclosure is directed to a method of measuring or detecting glyphosate in a sample. The method may include using a biosensor element 18 for measuring or detecting glyphosate in a sample. In one embodiment, the method may include using a low-cost portable device 10 that comprises a fluorescently labeled glyphosate binding protein as the biosensor element 18.

In some embodiments, the PhnD177NA construct conjugated with a fluorophore may act as the biosensor element 18. In some embodiments, such a biosensor element 18 may lack specificity for detecting glyphosate. While the PhnD177NA construct may bind with sufficient affinity to glyphosate, it may also bind to a number of other molecules with a similar structure. Such binding may potentially result in a false positive test if the biosensor element 18 binds to one of the other ligands it recognizes in the absence of glyphosate. Of the ligands that can interfere with the PhnD177NA construct, the most notable molecule is inorganic phosphate. While typical phosphate concentrations in water or soil are usually too low to interfere with the PhnD177NA construct phosphate concentrations may be high in heavily fertilized soils.

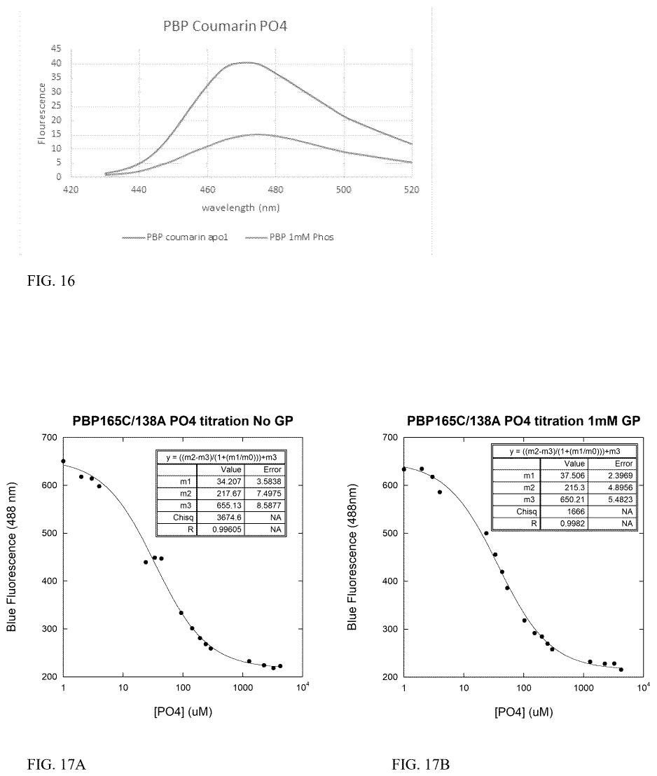

In some embodiments, to circumvent false positive tests due to phosphate contamination in samples, E. coli phosphate binding protein was developed as a control biosensor element 19 for phosphate. Previous work shows that attaching the fluorophore Coumarin at position 164 results in a large change in fluorescence when the E. coli phosphate binding protein binds to phosphate. However, E. coli phosphate binding protein exhibits a very high affinity for phosphate. Therefore, a PBP variant (PBP164C/138A) was engineered such that preserves the variant preserved the same large change in fluorescence and could detect phosphate in the micromolar range as shown in FIG. 16. The PBP variant (PBP164C/138A) has a dissociation constant (Kd) of about 35 μM for phosphate. Importantly, phosphate binding is not affected by the presence of glyphosate as shown in FIGS. 17A-17B. FIG. 17A is graph showing the titration of curve of PBP164C/138A in the absence of glyphosate. FIG. 17B is a graph showing the titration curve of PBP164C/138A in the presence of 1 mM glyphosate (m1: Kd in micromolar, m2: final fluorescence value, m3: initial fluorescence value). The results indicate that the presence of glyphosate does not interfere with the control biosensor element 19 for phosphate.

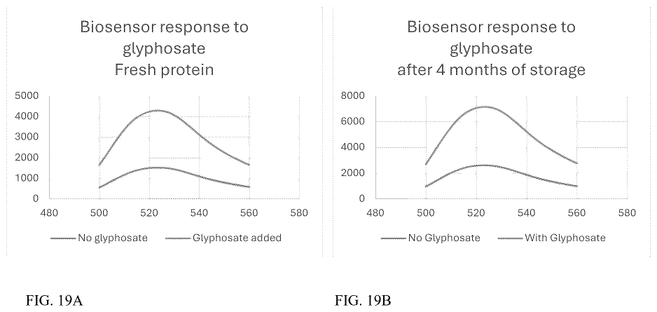

Since the glyphosate biosensor element 18 comes from E. coli, a mesophilic bacterial species, as with most proteins, the biosensor element 18 may be prone to denaturation over time. To develop the biosensor element 18 into a practical field deployable test, the stability of the protein must be addressed. In particular, the shelf life of the protein may be improved for better shipping and storage over several months. The fluorescence change associated with binding decreases over time when the biosensor element 18 is refrigerated in a buffer solution. Presumably, as more and more of the molecules in the biosensor element 18 denature, fewer of them respond to ligand binding resulting in progressively poorer performance. To address this issue, different lyophilization conditions to store the biosensor element 18 over long periods of time at room temperature were tested. Of the conditions tested, it was observed that lyophilization greatly expands the lifetime of the biosensor element 18. Adding 10% sucrose further enhances the performance of the biosensor element 18, showing that the biosensor element 18 can function robustly even after 4 months of storage at room temperature, as shown in FIGS. 19A-19B. Change in fluorescence of the glyphosate biosensor of fresh protein (FIG. 19A) is preserved for at least 4 months when the protein is lyophilized in the presence of sucrose (FIG. 19B).

### EXAMPLES AND METHODOLOGY

### Example 1: Testing Samples Containing Glyphosate

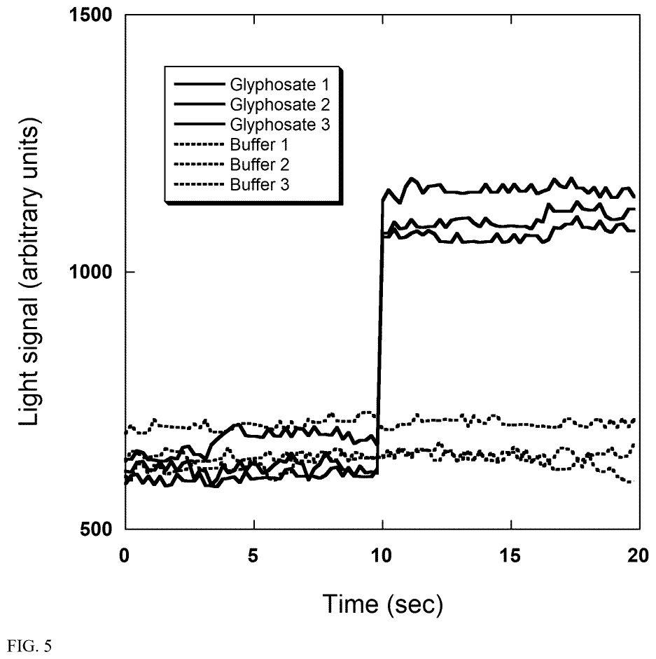

To confirm that the device is capable of detecting glyphosate, several tests were run by adding either working buffer containing glyphosate or working buffer alone to the biosensor. In each test 50 μL of the sample with or without glyphosate was added to 3 mL of biosensor at a concentration of about 300 nM. The final concentration of glyphosate in the positive samples was about 170 μM. The results illustrate that each of the samples containing glyphosate showed a large increase in fluorescence compared to the blank baseline, whereas samples with glyphosate showed no difference (see FIG. 5). In the case of samples containing glyphosate, the red LED illuminated after each test, whereas in the samples containing no glyphosate the green LED illuminated.

### Example 2: Testing Water Samples with Commercially Available Herbicides

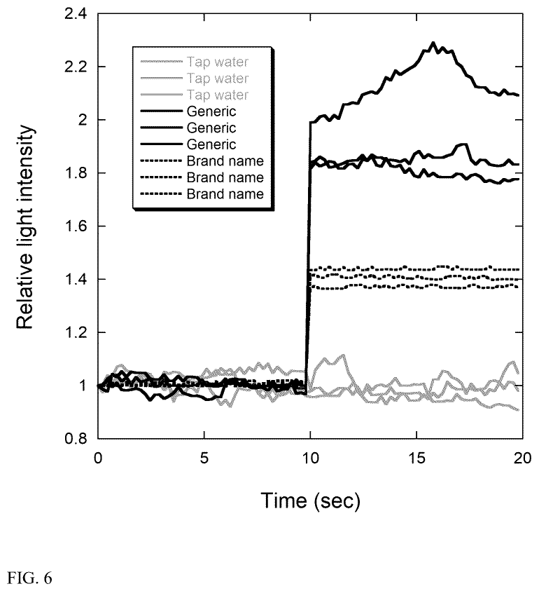

In order to show that the device can function in a real-life setting, the ability of the device to detect glyphosate in commercially available herbicides was evaluated. Often, these products contain other ingredients, which may potentially interfere with the sensing process or affect protein function. Tests included 2 products—a brand name weed killer (RoundUp Concentrate Plus) with 18% glyphosate isopropyl amine salt and a generic weed killer (Compare N Save Concentrate) with 41% glyphosate. Each product was diluted in tap water, according to the manufacturer's instructions, and 50 μL of each of the delusions were used for testing. Tap water was tested as a control. The results illustrate that weed killers produce positive tests, significantly increasing the fluorescence compared to the blank baseline. The brand-name weed killer showed about a 40% increase in fluorescence, whereas the generic brand showed about a doubling in the florescence output (see FIG. 6). No change was observed for tap water.

### Example 3: Testing Soil Samples, Containing Commercial Herbicides

Since there is widespread use of glyphosate on soils in residential, commercial, and farm settings, it is important that the device is able to test soil samples for the presence of glyphosate. Soil testing presents a different challenge as debris can cause turbidity within the test, which can interfere with the passing of the light through the device and/or cause light scattering, thereby obscuring the results. To overcome this challenge, an extract and filter approach was devised.

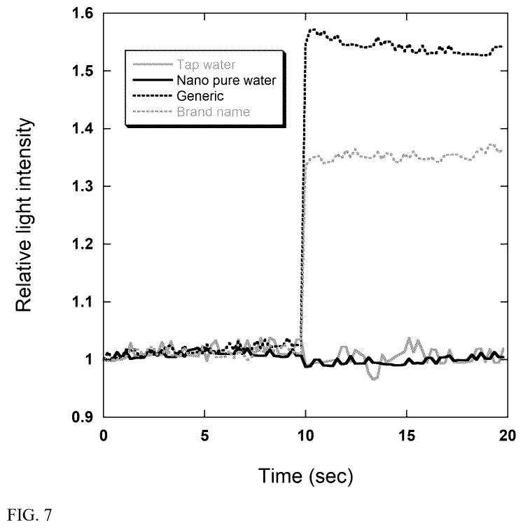

First, 10 grams of organic soil was sprayed with 10 mL of nano pure water (polished water), tap water, diluted, name brand, herbicide, or diluted generic herbicide. The soil samples were left overnight. Then approximately 1 gram of each soil sample was collected and mixed with 1 mL of working buffer in a 10 mL syringe. The mixture was then pushed through a 0.22 μm syringe filter. One drop from each sample (~35 μL) was added to a cuvette containing 300 nM of the biosensor in 3 mL of working buffer within the device. After collecting a baseline blank and sample addition, the results illustrate that both nano pure and tap water exhibit no change in fluorescence whereas the samples isolated from soils treated with the brand-name or the generic herbicide showed significant increases in fluorescence registering positive tests (see FIG. 7).

To confirm that the detected changes in fluorescence are indeed due to the interaction between the biosensor and glyphosate in the suspected samples, several controls were tested. First, a sample containing glyphosate in the absence of the protein biosensor was tested. No change in fluorescence was observed, indicating that glyphosate by itself does not change the light output and that the protein is required for detection (see FIG. 8). The specificity of the biosensor was tested by adding different ligands to the protein. No change in fluorescence was observed when the sugar maltose was added. These results illustrate that the protein binds specifically to glyphosate, and that this binding event triggers a detectable change in fluorescence.

### Example 4

A device 30 with more than one slot 28 with the biosensor elements 18 and the control biosensor element 19 was used to determine if sample that is identified as positive for glyphosate was contaminated with phosphate. The device 30 included dual glyphosate/phosphate biosensor elements 18.19 to determine if sample that is identified as positive for glyphosate was contaminated with phosphate. First, a 3 mL solution containing both PhnD177NA labeled with AF488 and PBP164C/138A labeled with Coumarin was placed in a cuvette inside the device 30. Second, a baseline was collected for each protein. A UV LED (372 nm) was used to excite the phosphate biosensor, and emission was collected in the 488 nm (blue) channel. Then a blue LED (453 nm) was used to excite the glyphosate biosensor, and emission was collected in the 515 nm (green) channel. Then a suspected sample was added to the cuvette, and the measurements were repeated. Finally, the processor was used to compare the data from both channels before and after addition of the sample.

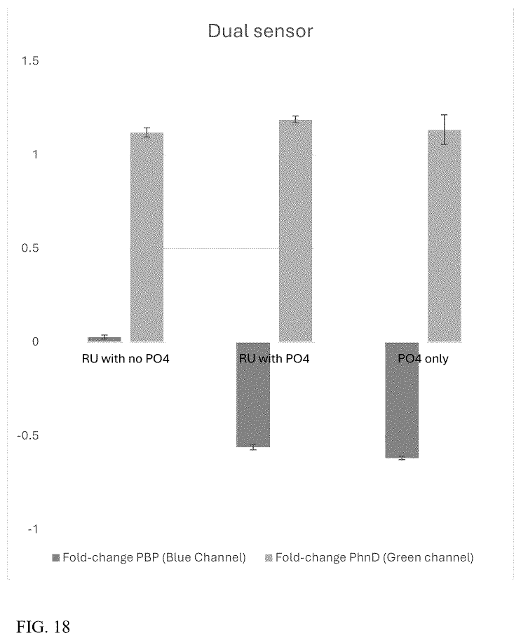

Unlike the glyphosate sensor which exhibits an increase in fluorescence upon ligand binding, the phosphate biosensor shows a decrease in fluorescence when phosphate binds. If only glyphosate is found in the sample, then an increase in the green channel is observed with no change in the blue channel. But since phosphate binds to both biosensors, a sample containing only phosphate or both phosphate and glyphosate would trigger changes in both channels. While this strategy does not distinguish between the presence of only phosphate or a mixture between phosphate and glyphosate, use of both glyphosate/phosphate proteins as the biosensor element 18 has a clear advantage over the single sensor strategy. As shown in FIG. 18, use of a dual sensor can identify if there is phosphate contamination and report a possible false positive requiring further analysis. The change in fluorescence in the PBP (blue channel) and the PhnD (green channel) is plotted in response to the presence of RoundUp only (RU with no PO4), both RoundUp and phosphate present (RU with PO4), or only phosphate (PO4 only).

The figures provided herein are not necessarily to scale, although a person skilled in the art will recognize instances where the figures are to scale and/or what a typical size is when the drawings are not to scale. While in some embodiments movement of one component is described with respect to another, a person skilled in the art will recognize that other movements are possible. Additionally, a number of terms may be used throughout the disclosure interchangeably but will be understood by a person skilled in the art. Further, to the extent features, sides, or steps are described as being “first” or “second,” such numerical ordering is generally arbitrary, and thus such numbering can be interchangeable. Still further, in the present disclosure, like-numbered components of various embodiments generally have similar features when those components are of a similar nature and/or serve a similar purpose. Lastly, the present disclosure includes some illustrations and descriptions that include prototypes, bench models, or experimental design. A person skilled in the art will recognize how to rely upon the present disclosure to integrate the techniques, systems, devices, and methods provided for into a product in view of the present disclosures.

While the concepts of the present disclosure are susceptible to various modifications and alternative forms, specific exemplary embodiments of the disclosure have been shown by way of example. It should be understood, however, that there is no intent to limit the concepts of the present disclosure to the particular disclosed forms; the intention is to cover all modifications, equivalents, and alternatives falling within the spirit and scope of the invention as defined by the claims. Although this disclosure refers to specific embodiments, it will be understood by those skilled in the art that various changes in form and detail may be made without departing from the subject matter set forth in the accompanying claims.

1. A device for detecting glyphosate in a sample, comprising:
a fluorescently labeled glyphosate binding protein as a biosensor element,
a first excitation source operable to excite the biosensor element at a first wavelength,
a light detector configured to collect emitted light, and
a processor configured to process any difference in fluorescence before and after a sample is added to the biosensor element.

2. The device of claim 1, wherein the device is portable and self-contained.

3. The device of claim 1, wherein the device provides a binary result indicating the presence or absence of glyphosate.

4. The device of claim 1, wherein the device is configured to operate as a miniature fluorometer.

5. The device of claim 1, wherein the biosensor element comprises E. coli bacterial periplasmic binding protein, PhnD.

6. The device of claim 1, wherein the biosensor element comprises E. coli bacterial periplasmic binding protein, PhnD, in which a cysteine mutation is moved to position 17 and wherein the biosensor element further comprises a C-terminal HSV tag followed by a 6-histidine tag.

7. The device of claim 5, wherein the protein is labeled with AlexaFluor488.

8. The device of claim 1, further comprising a control biosensor element configured to determine a concentration of phosphate in the sample.

9. The device of claim 1, further comprising a second excitation source operable to excite the biosensor element at a second wavelength.

10. The device of claim 1, operable to enable simultaneous detection of fluorescence at multiple wavelengths.

11. A method for detecting glyphosate, comprising:
adding a sample to a portable device comprising a fluorescently labeled glyphosate binding protein,
exciting the protein with an appropriate wavelength,
collecting emitted light from the protein through a light detector,
processing the collected light to determine any difference in fluorescence before and after a sample is added, and
providing a binary result indicating the presence or absence of glyphosate.

12. The method of claim 7, wherein the protein comprises E. coli bacterial periplasmic binding protein, PhnD.

13. The method of claim 7, wherein the protein comprises E. coli bacterial periplasmic binding protein, PhnD, in which a cysteine mutation is moved to position 17 and wherein the protein further comprises a C-terminal HSV tag followed by a 6-histidine tag.

14. The method of claim 8, wherein the protein is labeled with AlexaFluor488.

15. The method of claim 7, wherein the protein is configured to change fluorescence as a function of glyphosate concentration in the sample.

16. The method of claim 11, further comprising using a second protein to determine a concentration of phosphate in the sample.

17. The method of claim 16, wherein the second protein is PBP164C/138A.

18. The method of claim 11, further comprising providing a first visual indicator when the sample comprises glyphosate and second visual indicator when the sample does not comprise glyphosate.
